(19) United States
(12) Patent Application Publication
(10) Pub. No.: US 2025/0262021 A1
(43) Pub. Date: Aug. 21, 2025

(54) CANNULA AND STYLET REVERSER

(71) Applicant: Mayo Foundation for Medical Education and Research, Rochester, MN (US)

(72) Inventors: Christine U. Lee, Byron, MN (US); Susheil Uthamaraj, Menomonee Falls, WI (US); John J. Lau, Smyrna, GA (US)

(21) Appl. No.: 19/055,401

(22) Filed: Feb. 17, 2025

Related U.S. Application Data
(60) Provisional application No. 63/554,768, filed on Feb. 16, 2024.

(51) Int. Cl. A61B 90/00; A61N 5/10

(52) U.S. Cl. CPC A61B 90/39; A61N 5/1007; A61B 2090/3908; A61B 2090/3987; A61N 2005/1012; A61N 2005/1024

(57) ABSTRACT

Tissue markers may be implanted percutaneously through a needle into soft tissue to locate a site of a procedure at a later date. A medical implantation device for percutaneously implanting a tissue marker can include a housing extending from a proximal end to a distal end. The device may also include an obturator coupler disposed at the distal end of the housing to couple an obturator to the housing, and a guide track extending from a first track opening disposed at a distal end of the track toward a proximal end of the housing. The device can further include an actuator to be received within the guide track, where the actuator includes a cannula coupler disposed at a distal end of the actuator to couple a cannula to the actuator. The device can also include a latch arm configured to lock a position of the actuator along the guide track.

### CROSS-REFERENCE TO RELATED APPLICATIONS

This application claims the benefit of U.S. Provisional Patent Application Ser. No. 63/554,768, filed on Feb. 16, 2024, and entitled “CANNULA AND STYLET REVERSER,” which is herein incorporated by reference in its entirety.

### BACKGROUND

Tissue markers or localizers are often implanted within soft tissue, such as breasts, during or after a biopsy or other invasive procedure to allow medical professionals to locate the site of the procedure at a later time. Present methods of implanting tissue markers provide unpredictable marker placement in tissue. Conventional devices currently utilized to implant a marker into soft tissue, include an obturator or stylet that is axially movable through a fixed hollow cannula. Traditionally, the movable obturator is configured to translate within the fixed cannula to contact and push the tissue marker disposed therein through a distal opening of the cannula and into the soft tissue. Such methods lead to unpredictable marker placement, due to the variable local soft tissue forces, blood, and marker features.

In some disadvantageous instances, presently available implantation devices are incapable of implanting brittle or otherwise fragile medical devices. For example, the force required to push a medical device through the distal opening of the fixed cannula of traditional implantation devices can potentially break delicate medical devices or markers.

Finally, conventional implantation devices are preloaded with the marking device and operated by a sliding switch that pushes the marking device out of the cannula. There is no capacity for refined deployment of the marking device out of the cannula. As the marking device exits the cannula, the final position of the marking device is dependent on heterogeneous densities in the local tissue environment. A marking device can be deployed from a more traditional introducer (cannula with a sharp stylet), but this requires a practitioner to use two hands to properly implant a medical device, leading to implanting procedures that require multiple practitioners. In many cases, the practitioner implanting the medical device may also require the guidance of an imaging device, such as an ultrasound, to properly implant the medical device in an intended location. In such cases, a single practitioner will simultaneously operate both the implantation device and an imaging device. Present implantation devices that push the marking device out of the cannula do not control for tissue heterogeneity resulting in inaccurate or random positioning of the marking device as it leaves the needle.

As such, there remains a need for methods and systems to reliably implant medical devices into soft tissue at a predictable location.

### SUMMARY OF THE DISCLOSURE

According to an aspect of the present disclosure, a medical implantation device is provided. The medical implantation device includes a housing, the housing extending from a proximal end to a distal end. The device includes an obturator coupler arranged at the distal end of the housing to couple an obturator to the housing. The device includes a guide track extending along a length from a first track opening at a distal end of the guide track toward a proximal end of the housing. The device includes an actuator to be received within the guide track such that the actuator is translatable along the length of the guide track, the actuator including a cannula coupler arranged at a distal end of the actuator to couple a cannula to the actuator. The device includes a latch arm to lock a position of the actuator along the guide track.

According to other aspects of the present disclosure, the medical implantation device may include one or more of the following features. The latch arm may be configured to limit translation of the actuator along the length of the guide track. The latch arm may be rotatable about a hinge between a closed position and an open position to selectively permit the actuator to be axially translated relative to the length of the guide track. The actuator may include a first set of teeth. The latch arm may include a second set of teeth configured to mate with the first set of teeth to limit movement of the actuator. The guide track may be shaped to limit rotation of the actuator. The guide track may be integrally formed in the housing. The actuator may include a grip. The obturator coupler may be coaxial with the cannula coupler. When an obturator is coupled to the obturator coupler and a cannula is coupled to the cannula coupler, translation of the actuator towards the proximal end of the housing may cause the obturator to be received within the cannula. The obturator coupler may be integrally formed within the housing. The device may be composed of a light-weight material. The light-weight material may comprise a plastic. The device may be sterilizable and reusable. The cannula coupler may define a washer having a washer slot configured to receive the proximal end of the cannula.

According to another aspect of the present disclosure, a device for implanting a medical device into a patient is provided. The device includes a housing having a proximal end and a distal end. The device includes an obturator removably coupled to the distal end of the housing. The device includes an actuator slidably coupled to the housing and translatable along the housing parallel to an implantation axis. The device includes a cannula removably coupled to a distal end of the actuator, wherein the cannula is configured to receive the obturator and the medical device, and wherein translation of the actuator causes the cannula to slide over the obturator to release the medical device through a distal opening of the cannula.

According to other aspects of the present disclosure, the device for implanting a medical device into a patient may include one or more of the following features. The housing may comprise a guide track to receive and retain the actuator. The guide track may be configured to guide translation of the actuator and limit radial movement or rotation of the actuator relative to the implantation axis. The device may further comprise a latch arm configured to lock the actuator and limit axial movement of the actuator relative to the implantation axis. The latch arm may be rotatable about a hinge between a closed position and an open position to selectively permit the actuator to be axially translated relative to the implantation axis. The latch arm may be configured to limit distal movement of the actuator while permitting proximal movement of the actuator. The housing may comprise a housing receiver configured to removably couple a proximal end of the obturator. The housing receiver may comprise an inner volume defined by a first wall and a second wall connected by sidewalls. The obturator may comprise an obturator knob configured to be received and retained by the inner volume of the housing receiver. The obturator knob may be configured to contact the second wall to limit proximal translation of the obturator relative to the housing during operation. The actuator may comprise an actuator receiver configured to removably couple a proximal end of the cannula. The actuator receiver may define a washer having a washer slot configured to receive the proximal end of the cannula. The cannula may comprise a cannula knob configured to be received and retained by the washer slot of the actuator receiver. The medical device may be one of a tissue marker, a localizer, or a radioactive seed. The obturator may be configured to maintain a position of the medical device within the cannula until the actuator is translated to release the medical device through the distal opening of the cannula.

According to another aspect of the present disclosure, a device for implanting a medical device into a patient is provided. The device includes a housing having a proximal end and a distal end. The device includes an actuator slidably coupled to the housing and translatable along the housing parallel to an implantation axis. The device includes a guide track disposed on the housing and configured to receive and retain the actuator, wherein the guide track is shaped to limit radial movement and rotation of the actuator relative to the implantation axis. The device includes a latch arm coupled to one of the housing or the actuator, the latch arm being movable between a closed position and an open position, wherein in the closed position, the latch arm engages with the other of the housing or the actuator to limit axial movement of the actuator relative to the implantation axis, and wherein in the open position, the latch arm allows the actuator to translate along the guide track.

According to other aspects of the present disclosure, the device for implanting a medical device into a patient may include one or more of the following features. The guide track may comprise a pair of rails extending parallel to the implantation axis along a track sidewall of the housing. The latch arm may comprise arm teeth configured to engage with corresponding teeth on the other of the housing or the actuator. The latch arm may be configured to limit distal movement of the actuator while permitting proximal movement of the actuator when in the closed position. The housing may comprise a distal guide opening configured to receive a proximal end of the actuator for assembly of the device. The guide track may comprise a cap opposite a track sidewall, the cap connecting a pair of rails and configured to mitigate radial translation of the actuator relative to the implantation axis. The guide track may comprise a T-shaped cross-section configured to receive a complementary T-shaped portion of the actuator. The latch arm may be biased towards the closed position by a spring mechanism.

### BRIEF DESCRIPTION OF THE DRAWINGS

FIG. 1 is a perspective view of an implantation device, in accordance with some non-limiting examples of the disclosed subject matter.

FIG. 2 is a side view of the device of FIG. 1.

FIG. 3 is an exploded view of the device of FIG. 1.

FIG. 4A-4C are perspective views of the implantation device of FIG. 1 at different extension lengths.

FIG. 5 is a perspective view of an implantation device, in accordance with some non-limiting examples of the disclosed subject matter.

FIG. 6 is a front view of the device of FIG. 5.

FIG. 7 is a cross-section of the device of FIG. 6, taken at VI-VI.

FIG. 8 is a perspective view of the implantation device of FIG. 5 in an extended configuration.

FIG. 9 is a perspective view of the implantation device of FIG. 5, including a latch arm in an open position.

FIG. 10 is a top view of the device of FIG. 5.

FIG. 11 is a bottom view of the device of FIG. 5.

FIG. 12 is a perspective view of an implantation device, in accordance with some non-limiting examples of the disclosed subject matter.

FIG. 13 is a perspective view of an implantation device, in accordance with some non-limiting examples of the disclosed subject matter.

FIG. 14 is a perspective view of an implantation device, in accordance with some non-limiting examples of the disclosed subject matter.

FIG. 15 is a perspective view of an implantation device having an ergonomic housing, in accordance with some non-limiting examples of the disclosed subject matter.

FIG. 16 is a side view of an implantation device having an ergonomic housing, in accordance with some non-limiting examples of the disclosed subject matter.

FIGS. 17A and 17B show example configurations of a latch arm for releasably securing an actuator of the implantation device.

FIGS. 18A-18C show example configurations of an obturator holder and cannula holder, in accordance with some non-limiting examples of the disclosed subject matter.

FIG. 19 is a method for assembling an insertion device, in accordance with some non-limiting examples of the disclosed subject matter.

FIG. 20 is a method for inserting a medical device into a patient, in accordance with some non-limiting examples of the disclosed subject matter.

### DETAILED DESCRIPTION

Described herein are systems and methods for precisely implanting a soft tissue marker into a soft tissue of a patient. In accordance with various non-limiting examples, an improved implantation device removes the unpredictability and loss of control of the placement of the marker, as the marker exits a cannula into the tissue of the patient.

In some non-limiting examples, the implantation device can provide an improved instrument for implanting tissue markers. Additionally or alternately, the implantation device may be configured to implant various other medical devices into patient tissue. As will be described, the implantation device utilizes a fixed obturator, whose distal end contacts and moves a position of a medical device within a cannula. The cannula surrounds the obturator and the medical device and is configured to translate toward a proximal end of the implantation device to release the medical device through a distal cannula opening. The non-limiting example below is provided to illustrate the functionality and methods of use for the improved implantation device. As will be described further below, the improved implantation device can be configured to implant a variety of medical devices into a patient, including but not limited to tissue markers, localizers, radioactive seeds, or any other percutaneously implantable device. It is further noted that the improved implantation device can be used to percutaneously implant medical devices in both human and non-human patients (e.g., dogs, cats, horses, and other animals). The below description is but an example and should not limit the scope of the present disclosure.

The implantation device includes an obturator, a cannula, a housing, and an actuator. An implantation axis extends through a center of the obturator that is fixedly coupled to a distal end of the housing. The actuator is slidably coupled to the housing, and translatable along the housing parallel to the implantation axis. The obturator and the actuator may be of any length and may therefore be compatible with various lengths of introduces. The cannula is fixedly coupled to a distal end the actuator. During operation, a center of the cannula is aligned with the implantation axis, to allow a lumen of the cannula to receive the obturator. A medical device disposed in the lumen of the cannula can be implanted into a patient by sliding the actuator, and therefore the cannula, relative to the housing (e.g., toward a proximal end of the housing), and causing the obturator to push the medical device along the lumen and through a distal opening of the cannula.

In some non-limiting examples, the housing includes a guide track, configured to receive and retain the actuator. The guide track can be configured to guide the translation of the actuator, and therefore of the cannula. Furthermore, the guide track may be shaped to limit radial movement or rotation of the actuator, relative to the implantation axis. It is an advantage of the implantation devices described in the present disclosure that limiting the movement of rotation of the actuator prevents or otherwise reduces the implantation axis being offset during a procedure. In this way, inaccurate deployment of the medical device is mitigated because even minor rotations of the actuator, which could otherwise result in compounding errors in the placement of the medical device, are eliminated or otherwise reduced.

In some non-limiting examples, a latch arm can lock the actuator within the guide track, and limit axial movement of the actuator relative to the implantation axis. The latch arm can be rotatable about a hinge between a closed position and an open position to selectively permit the actuator to be axially translated relative to the implantation axis. In some non-limiting examples, the latch arm can be configured to limit distal movement of the actuator while permitting proximal movement of the actuator.

In some non-limiting examples, the actuator may include a grip. The grip may allow a user to operate the implantation device with a single hand. For example, the user may move the actuator via the grip using a thumb or other finger of a same hand that is holding the device. The grip may therefore allow an operator to control the implantation of a medical device into a patient with a single hand, ensuring the operator is able to control a secondary device with a free hand.

In some non-limiting examples, the housing and actuator of the implantation device may be reusable. For example, the housing and actuator may be sterilized using an autoclave or other sterilization methods (e.g., using dry heat, radiation (e.g., exposure to gamma radiation, electron beam radiation, ultraviolet radiation), ethylene oxide (EtO) gas, vaporized hydrogen peroxide, chlorine dioxide gas, vaporized peracetic acid, nitrogen dioxide, or the like), and used for multiple implantation procedures.

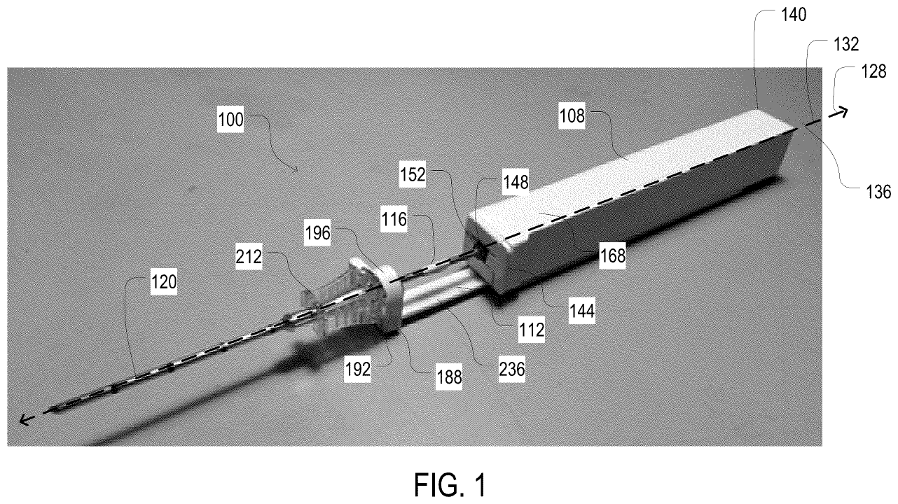

FIG. 1 illustrates a non-limiting example of an implantation device 100 that is configured to implant a medical device 104 (e.g., a tissue marker, localizer, radioactive seed, or other percutaneously implantable device) into a patient. The implantation device 100 may be used by itself or in conjunction with a secondary device (e.g., an imaging device) during an implantation procedure. The implantation device 100 includes a housing 108, an actuator 112, an obturator 116, and a cannula 120.

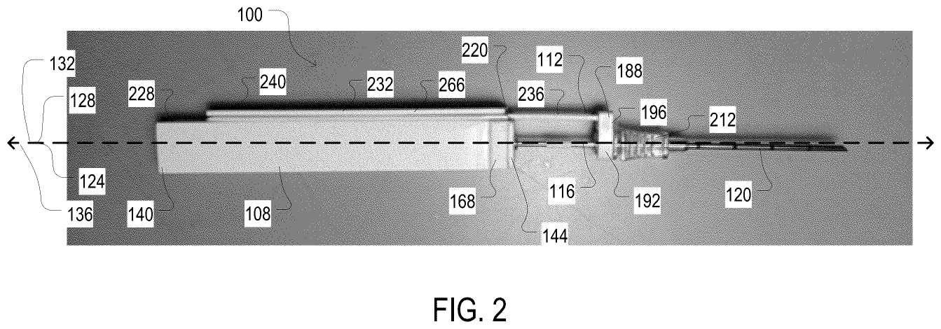

Referring to FIG. 2, the implantation device 100 may include an implantation axis 124. During operation an obturator center axis 128 and a cannula center axis 132 may be colinear with the implantation axis 124. That is, when an obturator 116 and a cannula 120 are arranged within the implantation device, they may be coaxial with each other. Furthermore, in some examples, a housing center axis 136 may be colinear with the implantation axis 124.

Referring again to FIG. 1, the implantation device 100 may include the housing 108 extending from a proximal housing end 140 to a distal housing end 144. The distal housing end 144 may include a housing receiver 148 configured to receive and retain the obturator 116. For example, the housing receiver 148 may be configured to removably couple a proximal obturator end 152. The obturator 116 may be removably coupled from the housing 108, to allow the housing 108 to be utilized for multiple implantation procedures. The housing receiver 148 may couple the proximal obturator end 152 via a snap-fit coupling, a threading, friction fit, adhesive, Luer taper, Tuohy Borst adapter, or any other suitable coupling arrangement.

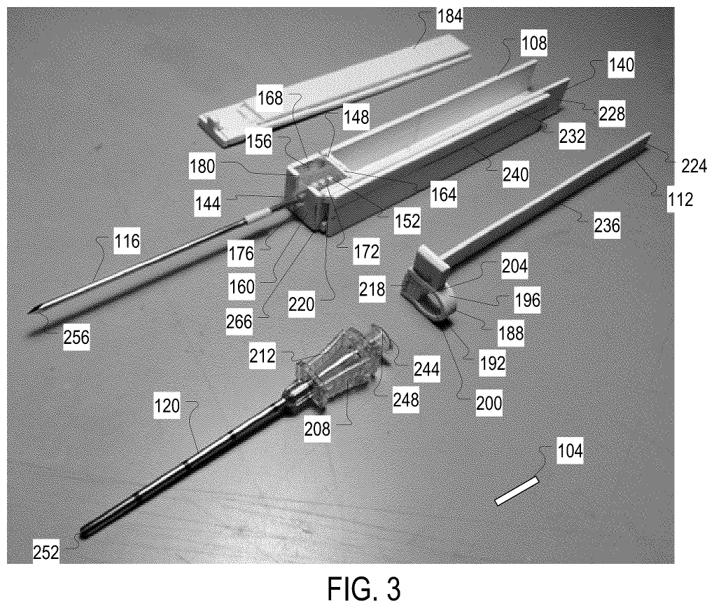

Referring to FIG. 3, the housing receiver 148 can include an inner volume 156 that may be defined by a first wall 160 and a second wall 164, connected by sidewalls 168. In some configurations, the first wall 160 may define a distal end of the housing receiver 148. Furthermore, the first wall 160 may define the distal housing end 144. In some configurations, the second wall 164 may be closer to the proximal housing end 140, than the first wall 160. Finally, the first wall 160 may be disposed directly opposite the second wall 164.

Still referring to FIG. 3, the inner volume 156 of the housing receiver 148 can be configured to receive the proximal obturator end 152. The obturator 116 includes an obturator knob 172 coupled to the proximal obturator end 152. The obturator knob 172, may be received and retained by the inner volume 156 of the housing receiver 148. The obturator knob 172 may contact the second wall 164 to limit proximal translation of the obturator 116 relative to the housing 108 during operation. Furthermore, the obturator knob 172 may contact one or more of the sidewalls 168 to limit radial rotation and translation of the obturator 116 relative to the implantation axis 124 during operation.

The obturator 116 may extend from the obturator knob 172, through an obturator slot 176 formed in the first wall 160. The obturator slot 176 may be U-shaped, including an obturator opening 180 that receives the obturator 116. The obturator slot 176 may define a smaller diameter than the obturator knob 172 to limit distal translation of the obturator 116 relative to the housing 108 during operation. In some non-limiting examples, the obturator slot 176 may taper toward the obturator opening 180, to provide a snap-fit connection to couple the obturator 116.

Still referring to FIG. 3, one of the sidewalls 168 defining the inner volume 156 may be a removable sidewall 184. In some non-limiting examples, the removable sidewall 184 may be configured to close the obturator opening 180 of the obturator slot 176. In such examples, the removable sidewall 184 may therefore ensure that the obturator 116 cannot exit the obturator slot 176 during operation.

Referring to FIG. 2, the actuator 112 of the implantation device 100 may be configured to translate axially relative to the implantation axis 124. In some non-limiting examples, the cannula 120 may be removably coupled to a distal actuator end 188, to allow the actuator 112 to translate the cannula 120 along the implantation axis 124.

Referring again to FIG. 3, the actuator 112 can include an actuator receiver 192 disposed at the distal actuator end 188. The actuator receiver 192 may define a washer 196 having a washer slot 200. The washer slot 200 may be U-shaped, including a cannula opening 204 to receive a proximal cannula end 208.

Referring again to FIG. 1, the cannula 120 includes a cannula knob 212 coupled to the proximal cannula end 208. As illustrated in FIG. 2, the cannula knob 212, may be received and retained by the washer slot 200 of the actuator receiver 192. In some non-limiting examples, the cannula knob 212 may include a recessed channel. The washer slot 200 may couple the recessed channel to mitigate translation of the cannula 120 relative to the actuator 112 during operation.

In some non-limiting examples, a diameter of the washer slot 200 may taper toward the cannula opening 204, to provide a snap-fit connection between the actuator 112 and the cannula knob 212. Furthermore, in some non-limiting examples, the cannula opening 204 may be closable by a tab 218 that extends across the cannula opening 204.

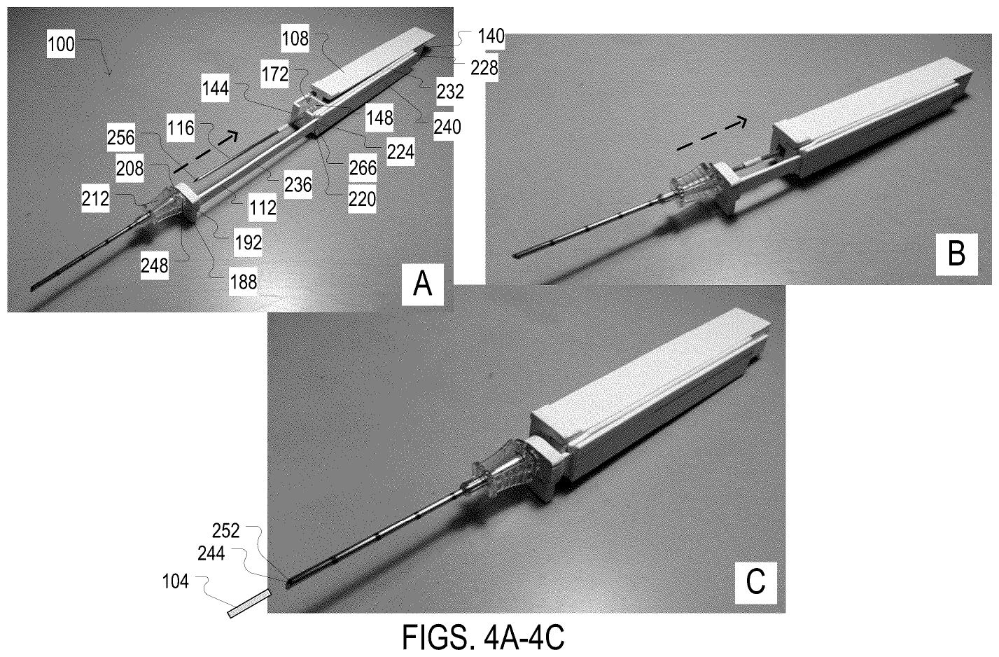

Referring to FIGS. 4A-4C, during operation the actuator 112, and therefore the cannula 120, may be translated relative to the housing 108. The housing 108 may include a guide track 266 configured to guide the translation of the actuator 112. The guide track 266 may include a distal guide opening 220. As illustrated in FIG. 4A, the distal guide opening 220 may be configured to receive a proximal actuator end 224 to assemble the housing 108 and the actuator 112. The actuator 112 may then be slid within the guide track 266 toward the proximal housing end 140.

The guide track 266 may extend from a track sidewall 228 (e.g., one of the sidewalls 168 of the housing 108), and may be defined by a pair of rails 232. The pair of rails 232 may extend parallel to the implantation axis 124 along the track sidewall 228. The guide track 266 may be configured to receive an actuator bar 236, that extends from the actuator receiver 192. The actuator bar 236 may slide between the pair of rails 232, to guide axial translation of the actuator 112 relative to the implantation axis 124. In some non-limiting examples, the guide track 266 may include a cap 240, opposite the track sidewall 228, connecting the pair of rails 232. The cap 240 may mitigate radial translation or rotation of the actuator 112, relative to the implantation axis 124, during operation. In some non-limiting examples, the guide track 266 may include fewer or more rails.

Still referring to FIGS. 4A-4C, the implantation device 100 is utilized to implant the medical device 104 in a tissue of a patient. The medical device 104 may be received, retained, and transported by a lumen 244 of the cannula 120 that extends from a proximal lumen opening 248 to a distal lumen opening 252. During operation as the cannula 120 is translated by the actuator 112 along the implantation axis 124 toward the proximal housing end 140, the proximal lumen opening 248 receives a distal obturator end 256. As the cannula 120 continues to be translated toward the proximal housing end 140, the distal obturator end 256 may contact the medical device 104 disposed within the lumen 244 to maintain a position of the medical device 104, until the proximal translation of the cannula 120 causes the release of the medical device 104 through the distal lumen opening 252. During insertion of the cannula 120 into a patient (not shown), the cannula 120 may pierce the patient tissue (not shown) and form a cannula pocket (not shown) in the patient tissue. During implantation of the medical device 104 into the patient (not shown), the proximal translation of the cannula 120 may release the medical device 104 into the resulting cannula pocket (not shown). In such embodiments, the operator of the implantation device 100 can be positive that the medical device 104 will be retained by the resulting cannula pocket in the patient tissue, leading to more accurate and more precise placement of the medical device 104 during implantation.

In some embodiments, once an implantation procedure is finished, the implantation device 100 may be disassembled. As described above, each of actuator 112, obturator 116, and cannula 120 can be removed from the housing 108. The individual components of the implantation device 100 can selectively be disposed, or cleaned for another implantation procedure. For example, the actuator 112 and the housing 108 can be sterilized using an autoclave or other sterilization technique, before being utilized for another implantation procedure.

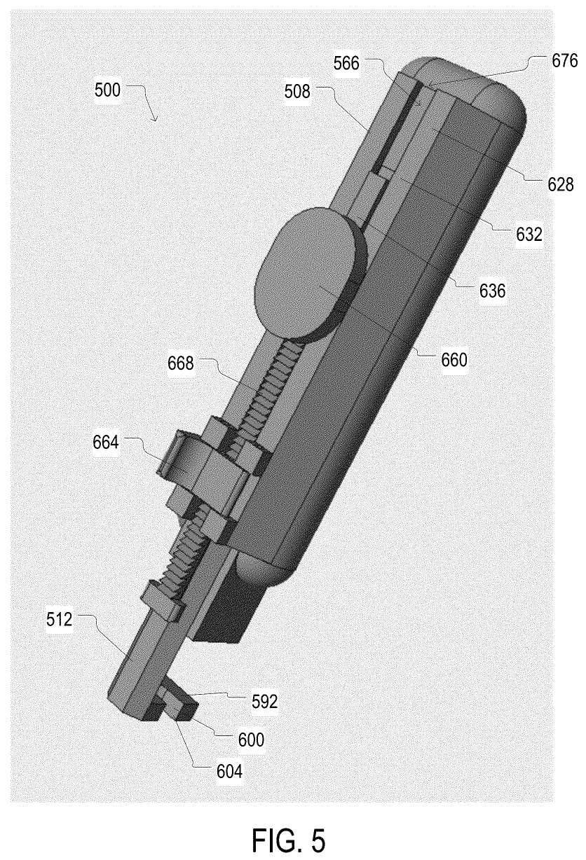

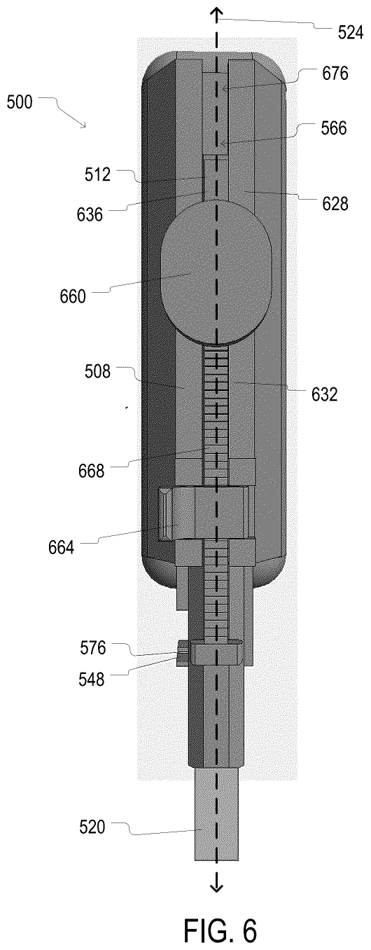

In some non-limiting examples, a locking mechanism may aid the securement of an actuator to a housing. For example, FIGS. 5-11 illustrate another embodiment of an implantation device 500 including an alternate actuator locking mechanism. The implantation device 500 may generally include similar features as the implantation device 500, including but not limited to a medical device 504, housing 508, an actuator 512, an obturator 516, a cannula 520, and an implantation axis 524. Furthermore, the implantation device 500 may include similar mechanisms to couple the various components together, including a housing receiver 548, an obturator slot 576, an obturator knob 572, an actuator receiver 592, a washer slot 600, a cannula knob 612, an actuator bar 636, a guide track 566, and rails 632. Thus, discussion of the implantation device 100 above can also generally apply to similar components of the implantation device 500. However, in some aspects, the implantation devices 100 and 500 may differ. In particular, the implantation device 500 includes an alternate guide track and actuator configuration.

Referring to FIG. 5, the actuator 512 may include a grip 660 disposed on the actuator bar 636. During operation, the grip 660 may extend away from the housing 108. The grip 660 may allow the actuator to be operated by a single finger (e.g., a thumb or index finger). For example, the grip 660 can aid a user to hold the implantation device 500 and translate the actuator 512 utilizing a single hand.

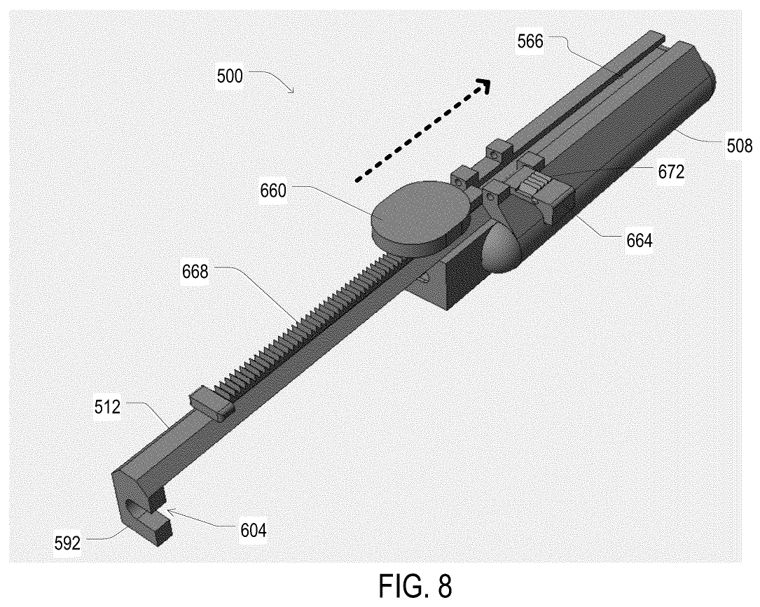

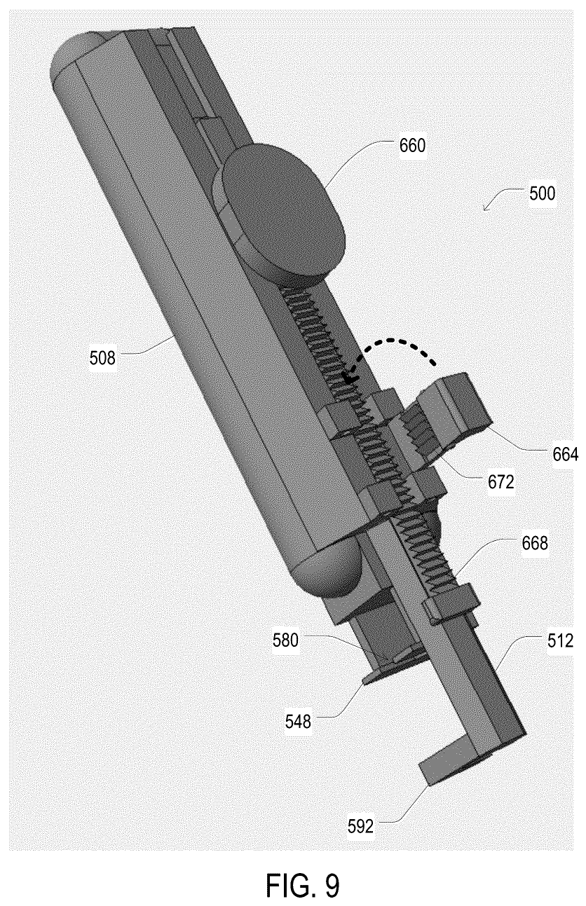

Referring to FIGS. 5, 8, and 9, the housing 508 may include a latch arm 664. The latch arm 664 may rotate on a hinge between a closed position (see FIG. 5) and an open position (see FIGS. 8 and 9). In the closed position, the latch arm 664 may extend over the guide track 566 to mitigate axial translation of the actuator 512 relative to the implantation axis 524. As will be described below, the latch arm 664 may couple the actuator 512 via a snap-fit coupling, a teethed coupling, friction coupling, clamp coupling, or any other suitable coupling arrangement. In the open position, the latch arm 664 may not extend over the guide track 566, or may not engage the actuator 512, to allow the actuator 512 to freely slide within the guide track 566.

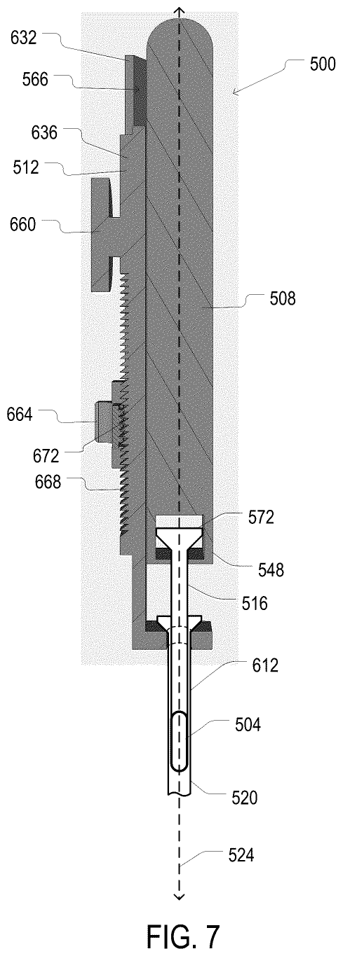

Referring to FIG. 7, the actuator 512 may include a plurality of actuator teeth 668 disposed on the actuator bar 636, configured to mitigate axial translation of the actuator 512 relative to the implantation axis during operation. When the actuator 512 is properly seated in the guide track 566, the actuator teeth 668 may extend away from the housing 508. As illustrated in FIG. 7, the latch arm 664 may include a plurality of arm teeth 672 configured to mate with the actuator teeth 668. In the closed position of the latch arm 664, the arm teeth 672 may mate with the actuator teeth 668 to mitigate axial translation of the actuator 512 relative to the implantation axis 524. In some non-limiting examples, the mating of the arm teeth 672 and the actuator teeth 668 may mitigate axial translation of the actuator 512 relative to the implantation axis 524, in a first direction (e.g., in a distal to proximal direction), while allowing the actuator 512 to freely slide along the guide track 566 in the second direction (e.g., opposite the first direction). In other non-limiting examples, the first direction is instead in a proximal to distal direction.

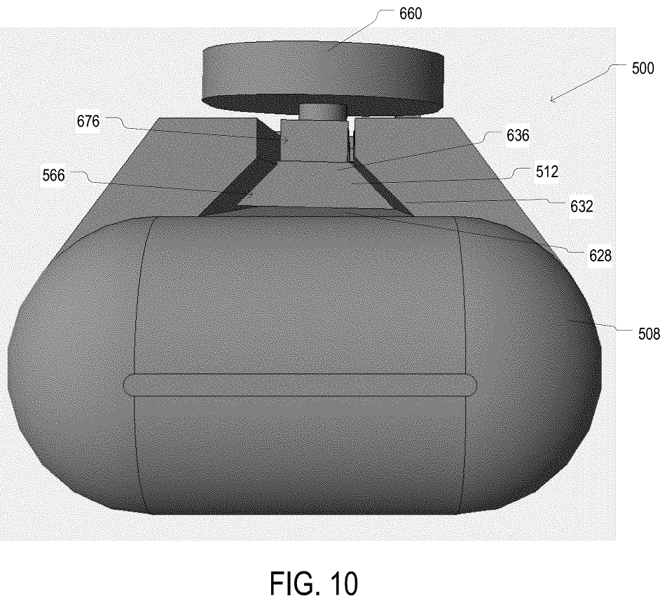

Referring to FIG. 10, the guide track 566 may be shaped to mitigate radial translation or rotation of the actuator 512 during operation. For example, the pair of rails 632 may extend from a track sidewall 628 at an oblique angle to form a guide track 566 that tapers in width (e.g., a distance between the two closest co-radial points on separate rails 632), as the pair of rails 632 extend from the track sidewall 628. The pair of rails 632 may extend toward each other at opposite oblique angles to form a triangular cross-sectional shape of the guide track 566. In some non-limiting examples, the angled rails 632 may not intersect, forming a gap 676 between the pair of rails 632. In some non-limiting examples, the gap 676 may account for radial projections disposed on the actuator bar 236, such as the grip 660 and the actuator teeth 668.

Still referring to FIG. 10, in some non-limiting examples, a width of the gap 676 between the pair of rails 632 may be less than a width of the actuator bar 636 to limit radial translation or removal of the actuator 512 from the guide track 566. In some non-limiting examples, the guide track 566 and the actuator bar 636 may define similar or identical cross-sectional shapes. The similar cross-sectional shapes of the actuator bar 636 and the guide track 566 may help to mitigate radial translation and rotation of the actuator bar relative to the implantation axis 524 during operation.

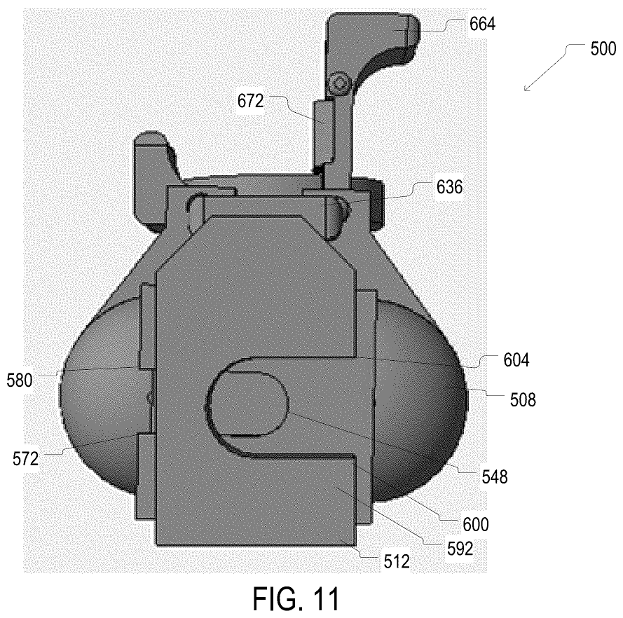

Referring to FIG. 11, a cannula opening 604 of the actuator receiver 592 and an obturator opening 580 of the housing receiver 548 may be oriented in different directions. As described above, during operation the obturator 516 may be disposed within the cannula 520. Therefore, radially translating or bending the cannula 520 relative to the implantation axis 524 may radially translate or bend the obturator 516 relative to the implantation axis 524, and vice versa. Accordingly, the differently oriented cannula opening 604 and obturator opening 580 may help to ensure the obturator 516 and the cannula 520 do not decouple from the cannula opening 604 and obturator opening 580. For example, the cannula 520 translating or bending toward the cannula opening 604 may contact the housing receiver 548 prior to exiting the cannula opening 604. Similarly, the obturator 516 translating or bending toward the obturator opening 580 may contact the actuator receiver 592 prior to exiting the obturator opening 580.

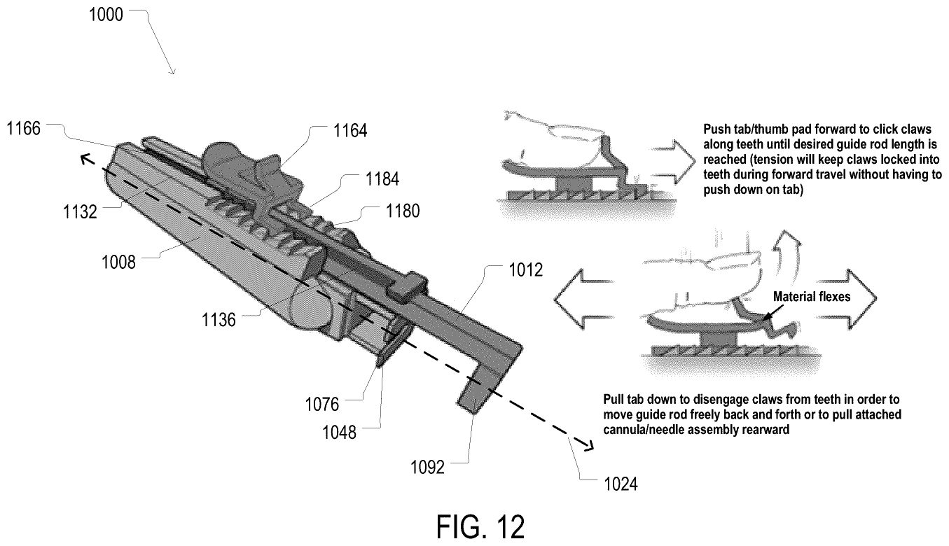

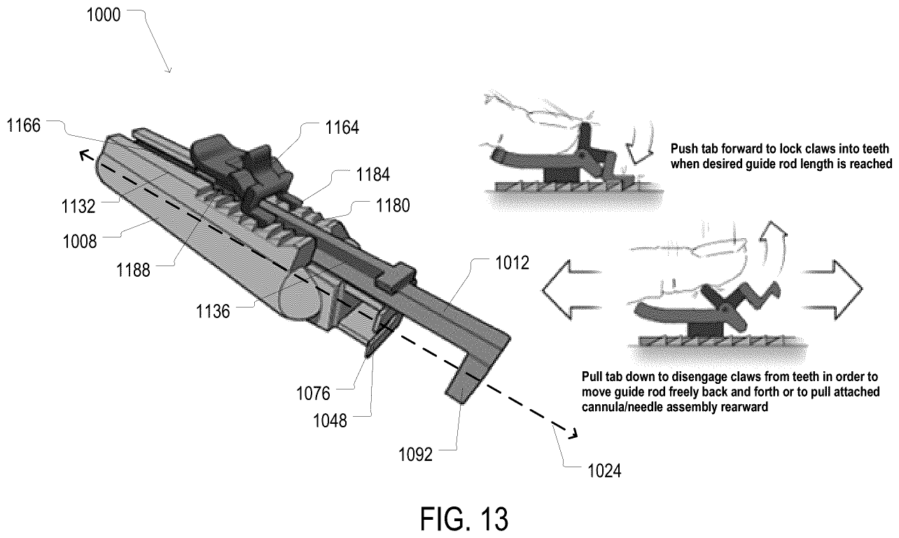

In some non-limiting examples, an alternate locking mechanism may aid the securement of an actuator to a housing. For example, FIGS. 12 and 13 illustrate another embodiment of an implantation device 1000 including an alternate actuator locking mechanism. The implantation device 1000 may generally include similar features as the implantation device 500, including but not limited to a housing 1008, an actuator 1012, an obturator (not illustrated), a cannula (not illustrated), and an implantation axis 1024. Furthermore, the implantation device 1000 may include similar mechanisms to couple the various components together, including a housing receiver 1048, an obturator slot 1076, an obturator knob (not illustrated), an actuator receiver 1092, a washer slot (not illustrated), a cannula knob (not illustrated), an actuator bar 1136, a guide track 1166, and a latch arm 1164. Thus, discussion of the implantation device 500 above can also generally apply to similar components of the implantation device 1000. However, in some aspects, the implantation devices 500 and 1000 may differ. In particular, the implantation device 1000 includes an alternate guide track and actuator configuration.

Referring to FIG. 12, the rails 1132 of the guide track 1166 may include rail teeth 1180. The rail teeth 1180 may extend opposite the housing 1008, and may be configured to engage and hold the actuator 1012 during operation.

Still referring to FIG. 12, the actuator 1012 may include the latch arm 1164 extending from the actuator bar 1136. The latch arm 1164 may include arm teeth 1184 that are configured to engage the rail teeth 1180 during operation. In some non-limiting examples, the latch arm 1164 may be a resilient member. The arm teeth 1184 of the latch arm 1164 may springily engage the rail teeth 1180 to maintain an axial position of the actuator 1012. The latch arm 1164 may be resiliently deformable to disengage the arm teeth 1184 from the rail teeth 1180. The latch arm 1164 may be resiliently deformed using a thumb or other finger, to allow an operator to control the actuator 1012 using a single hand, ensuring an operator is able to control a second device with their free hand.

Referring to FIG. 13, the latch arm 1164 may instead be rotated about a hinge 1188 to engage and disengage the arm teeth 1184 from the rail teeth 1180.

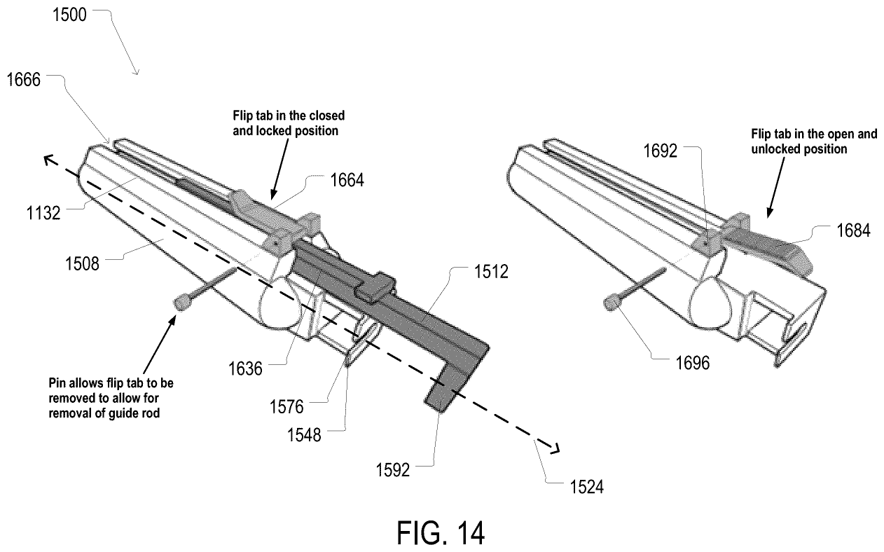

In some non-limiting examples, an alternate locking mechanism may aid the securement of an actuator to a housing guide track. For example, FIGS. 14 illustrate another embodiment of an implantation device 1500 including an alternate actuator locking mechanism. The implantation device 1500 may generally include similar features as the implantation device 1000, including but not limited to a housing 1508, an actuator 1512, an obturator (not illustrated), a cannula (not illustrated), and an implantation axis 1524. Furthermore, the implantation device 1500 may include similar mechanisms to couple the various components together, including a housing receiver 1548, an obturator slot 1576, an obturator knob (not illustrated), an actuator receiver 1592, a washer slot (not illustrated), a cannula knob (not illustrated), an actuator bar 1636, a guide track 1666, and a latch arm 1664. Thus, discussion of the implantation device 500 above can also generally apply to similar components of the implantation device 1500. However, in some aspects, the implantation devices 500 and 1500 may differ. In particular, the implantation device 1500 includes an alternate guide track and actuator configuration.

Referring to FIG. 14, the latch arm 1664 may be coupled to the guide track 1666 via a hinge 1692. The hinge 1692 may include a removable pin 1696 to allow the hinge 1692 and the latch arm 1664 to be removed from the housing 1508. The latch arm 1664 may be rotatable between an open position and a closed position. The latch arm 1664 may include arm teeth 1684 configured to engage the actuator 1512 in the closed position. The arm teeth 1684 may increase friction between the latch arm 1664 and a surface of the actuator 1512. In the open position of the latch arm 1664, the actuator 1512 may be free to slide along the guide track 1666.

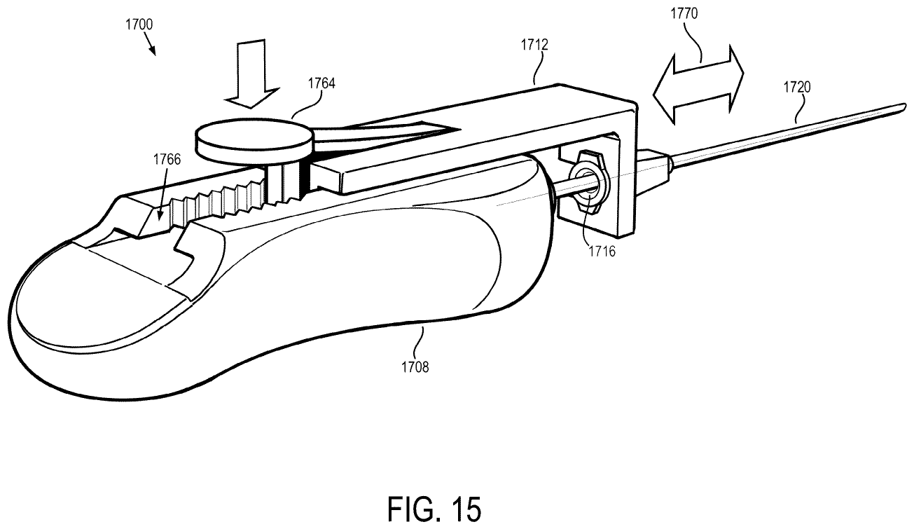

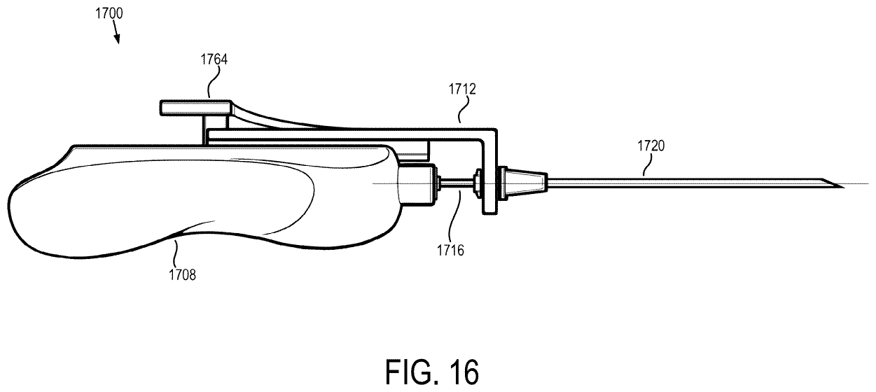

Referring now to FIGS. 15 and 16, an example implantation device 1700 according to some embodiments is shown. The implantation device 1700 includes a housing 1708, an actuator 1712, an obturator 1716, and a cannula 1720. In the illustrated example, the housing 1708 of the implantation device 1700 is ergonomically shaped. As such, the housing 1708 may facilitate the user operating the implantation device 1700 with a single hand. For example, the user may hold the housing 1708 comfortably in their hand based on the shape of the housing 1708, and may then move the actuator 1712 via a latch arm 1764 using a thumb or other finger of the same hand that is holding the implantation device 1700. The ergonomic shape of the housing 1708 and the positioning of the latch arm 1764 may therefore allow an operator to control the implantation of a medical device into a patient with a single hand, ensuring the operator is able to control a secondary device with a free hand. The ergonomics of the housing 1708 may take into account not only the shape of the housing 1708, but also the weight and grippability of the housing 1708. Advantageously, the ergonomics of the housing 1708 can minimize effects downstream at the cannula needle tip that may otherwise affect accurate placement of the medical device.

In some cases, the size and shape of the housing 1708 may be designed to accommodate different hand sizes. For instance, different housings 1708 may be constructed with different sizes and/or shapes. As a non-limiting example, one version of the housing 1708 may be sized as a “small” size, one version may be sized as a “medium” size, and one version may be sized as a “large” size. Advantageously, the same obturator and cannula hardware components may be used with different sized housing 1708 because the obturator 1716 and cannula 1720 are removably coupled to the housing 1708. In this way, a hospital or clinical site may have different housings 1708 available for users with different sized hands, and the same sized obturator and cannula hardware could be used with the different sized housings 1708. Since the housing 1708 may be made to be reusable, the interchangeability of the same obturator and cannula components with different sized housings 1708 is an advantage of the implantation devices described in the present disclosure.

The latch arm 1764 can lock the actuator 1712 within the guide track 1766, and limit axial movement of the actuator 1712 relative to the implantation axis. The latch arm 1764 can be rotatable about a hinge between a closed position and an open position to selectively permit the actuator 1712 to be axially translated relative to the implantation axis, as indicated by arrows 1770. In some non-limiting examples, the latch arm 1764 can be configured to limit distal movement of the actuator 1712 while permitting proximal movement of the actuator 1712.

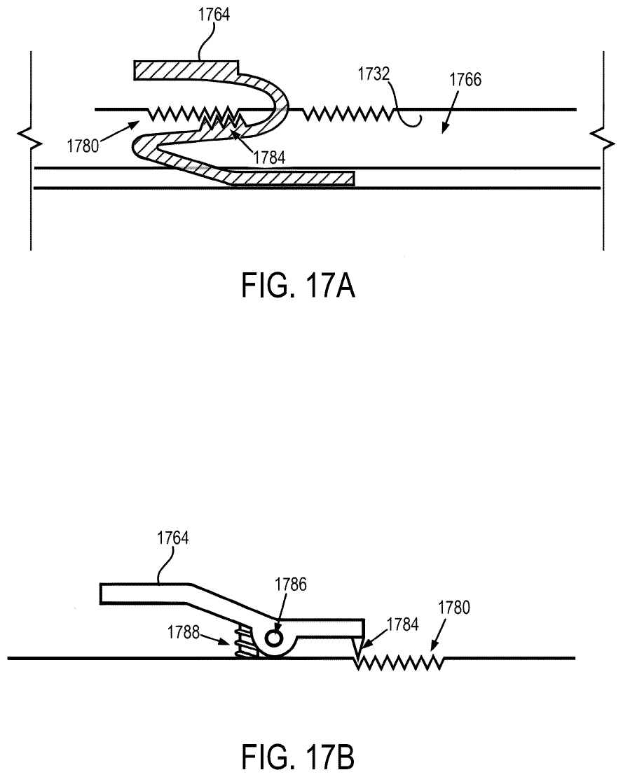

In some embodiments, such as those illustrated in FIG. 17A, the latch arm 1764 may include a hinged design (e.g., a flexible hinge) that engages with the guide track 1766 between the closed position and the open position. In the illustrated example, the rails 1732 of the guide track 1766 may include rail teeth 1780 that are arranged on an inner surface of the rails 1732. The rail teeth 1780 are configured to engage and hold the actuator 1712 during operation. The latch arm 1764 may include corresponding arm teeth 1784 that are configured to engage the rail teeth 1780 during operation. In some non-limiting examples, the latch arm 1764 may be a resilient member. The arm teeth 1784 of the latch arm 1764 may springily engage the rail teeth 1780 to maintain an axial position of the actuator 1712. The latch arm 1764 may be resiliently deformable to disengage the arm teeth 1784 from the rail teeth 1780. The latch arm 1764 may be resiliently deformed using a thumb or other finger, to allow an operator to control the actuator 1712 using a single hand, ensuring an operator is able to control a second device with their free hand. By way of example, depressing the latch arm 1764 will disengage the arm teeth 1784 from the rail teeth 1780, allowing translation of the actuator 1712 within the guide track 1766. When pressure from the latch arm 1764 is released the latch arm 1764 will spring back to its initial state based on the resilient construction of the latch arm 1764. As such, the arm teeth 1784 will again engage the rail teeth 1780 to lock the actuator 1712 into place.

As illustrated in FIG. 17B, in an alternative configuration the latch arm 1764 may instead be a hinged member that rotates about a pivot 1786. The latch arm 1764 may have one or more arm teeth 1784 that are configured to engage corresponding rail teeth 1780 on the guide track 1766. In the illustrated example, the rail teeth 1780 are on the top, outer surface of the guide track 1766. A spring 1788 biases the latch arm 1764 in its closed position, such that the arm teeth 1784 are engaged with the rail teeth 1780 to secure the actuator 1712 in a locked position. Depressing the latch arm 1764 disengages the arm teeth 1784 from the rail teeth 1780, thereby permitting translation of the actuator 1712. When pressure is released from the latch arm 1764, the spring 1788 again biases the latch arm 1764 in its closed position, such that the arm teeth 1784 again engage the rail teeth 1784 to lock the actuator 1712 into place. Alternatively, in lieu of the rail teeth 1780 a plurality of actuator teeth may be disposed on the actuator bar and configured to mitigate axial translation of the actuator 1712 relative to the implantation axis during operation.

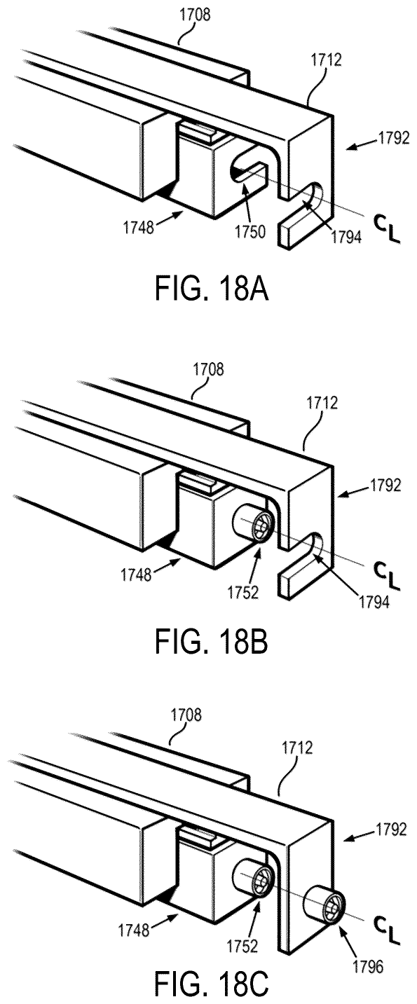

Referring now to FIGS. 18A-18C, example constructions of an obturator holder 1748 (e.g., obturator coupler, housing receiver) and cannular holder 1792 (e.g., cannula coupler, actuator receiver) that can be implemented in various configurations of the implantation devices described in the present disclosure. As described above, the obturator holder 1748 is coaxial with the cannula holder 1792.

The obturator holder 1748 (e.g., obturator coupler, housing receiver) is configured to receive and retain the obturator 1716 in the housing 1708. For example, the obturator holder 1748 may be configured to removably couple a proximal obturator end. The obturator 1716 may be removably coupled from the housing 1708 to allow the housing 1708 to be utilized for multiple implantation procedures. In the example illustrated in FIG. 18A, the obturator holder 1748 enables quick attachment of the obturator 1716 to the housing 1708 via a U-shaped slot 1750 formed in the obturator holder 1748. Additionally, the example construction illustrated in FIG. 18A provides for self-positioning of the obturator 1716. In the examples illustrated in FIGS. 18B and 18C, the obturator holder 1748 includes a Luer connector 1752.

The cannula holder 1792 (e.g., cannula coupler, actuator receiver) is configured to receive and retain the cannula 1720 in the actuator 1712. For example, the cannula holder 1792 may be configured to removably couple a proximal cannula end. The cannula 1720 may be removably coupled from the actuator 1712 to allow the actuator 1712 to be utilized for multiple implantation procedures. In the example illustrated in FIGS. 18A and 18B, the cannula holder 1792 enables quick attachment of the cannula 1720 to the actuator 1712 via a U-shaped channel 1794 formed in the cannula holder 1792. The cannula holder 1792 may be integrally formed with the actuator 1712. In the example illustrated in FIG. 18C, the cannula holder 1792 includes a Luer connector 1796.

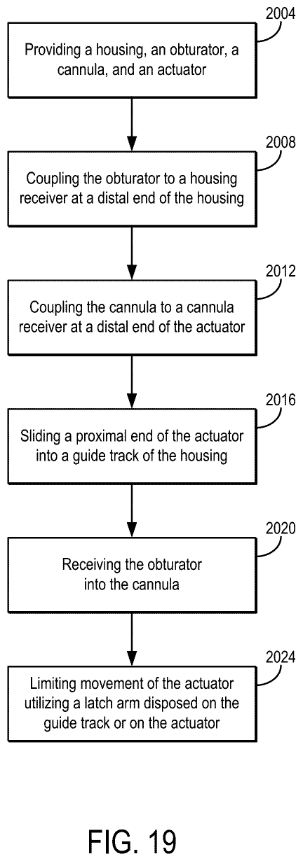

Referring now to FIG. 19, a method 2000 is illustrated for assembling an implantation device, which may include fewer or more steps than depicted. In some embodiments, the following steps are performed in any order. At a first step 2004, the method 2000 includes providing a housing, an obturator, a cannula, and an actuator. At a second step 2008, the method 2000 includes coupling the obturator to a housing receiver at a distal end of the housing. At a third step 2012, the method 2000 includes coupling the cannula to a cannula receiver at a distal end of the actuator. At a fourth step 2016, the method 2000 includes sliding a proximal end of the actuator into a guide track of the housing. At a fifth step 2020, the method 2000 includes receiving the obturator into the cannula. At a sixth step 2024, the method 2000 includes limiting movement of the actuator utilizing a latch arm disposed on the guide track or on the actuator.

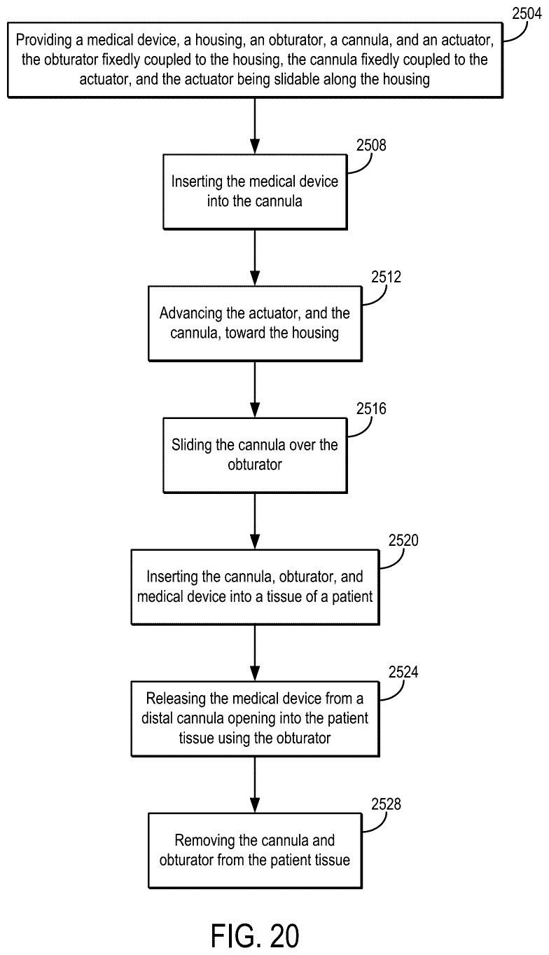

Referring now to FIG. 20, a method 2500 is illustrated for utilizing an implantation device, which may include fewer or more steps than depicted. In some embodiments, the following steps are performed in any order. At a first step 2504, the method 2500 includes providing a medical device, a housing, an obturator, a cannula, and an actuator, the obturator fixedly coupled to the housing, the cannula fixedly coupled to the actuator, and the actuator being slidable along the housing. At a second step 2508, the method includes 2500 inserting the medical device into the cannula. At a third step 2512, the method 2500 includes advancing the actuator, and the cannula, toward the housing. At a fourth step 2516, the method 2500 includes sliding the cannula over the obturator. At a fifth step 2520, the method 2500 includes inserting the cannula, obturator, and medical device into a tissue of a patient. At a sixth step 2528, the method 2500 includes releasing the medical device from a distal cannula opening into the patient tissue using the obturator. At a seventh step 2524, the method 2500 includes removing the cannula and obturator from the patient tissue.

The implantation device described above can be manufactured from any material. Preferably, the implantation device may be light-weight to reduce user fatigue. For example, the implantation device, or components thereof, can be composed of light-weight plastic, polymers, metals, metal alloys, or other materials. As a non-limiting example, the implantation device, or components thereof, can be composed of polyethylene, polypropylene, polyvinyl chloride (PVC), polyethylene terephthalate (PET), polycarbonate, polyurethane, acrylonitrile butadiene styrene (ABS), polymethyl methacrylate (PMMA), polyoxymethylene (POM) or acetal, polytetrafluoroethylene (PTFE), or the like. In some instances, the implantation device, or components thereof, can be manufactured using additive manufacturing techniques, such as 3D printing. In some other examples, the implantation device, or components thereof, can be composed of other materials, including stainless steel, titanium, metal alloys, ceramics, or the like. When using metals and metal alloys, in some cases light-weight metals and/or metal alloys may be used to maintain a lightweight, ergonomic design for the implantation device.

It is to be understood that the systems and methods described in the present disclosure are not limited in their application to the details of construction and the arrangement of components set forth in the preceding description or illustrated in the drawings. The disclosed systems and methods are capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings, and may also include fluid and electrical connections.

One or more embodiments are described and illustrated in the preceding description and accompanying drawings. These embodiments are not limited to the specific details provided herein and may be modified in various ways. Further, other embodiments may exist that are not expressly described herein. Also, functions described as being performed by multiple components may be consolidated and performed by a single component. Similarly, functions described herein as being performed by one component may be performed by multiple components in a distributed manner. Additionally, a component described as performing particular functionality may also perform additional functionality not expressly described herein. For example, a device or structure that is “configured” in a certain way is configured in at least that way, but may also be configured in ways that are not expressly listed.

1. A medical implantation device, comprising
a housing, the housing extending from a proximal end to a distal end;
an obturator coupler arranged at the distal end of the housing to couple an obturator to the housing;
a guide track extending along a length from a first track opening at a distal end of the guide track toward a proximal end of the housing;
an actuator to be received within the guide track such that the actuator is translatable along the length of the guide track, the actuator including a cannula coupler arranged at a distal end of the actuator to couple a cannula to the actuator; and
a latch arm to lock a position of the actuator along the guide track.

2. The medical implantation device of claim 1, wherein the latch arm is configured to limit translation of the actuator along the length of the guide track.

3. The device of claim 2, wherein the latch arm is rotatable about a hinge between a closed position and an open position to selectively permit the actuator to be axially translated relative to the length of the guide track.

4. The medical implantation device of claim 1, wherein the actuator includes a first set of teeth and the latch arm includes a second set of teeth configured to mate with the first set of teeth to limit movement of the actuator.

5. The medical implantation device of claim 1, wherein the guide track is shaped to limit rotation of the actuator.

6. The medical implantation device of claim 1, wherein the guide track is integrally formed in the housing.

7. The medical implantation device of claim 1, wherein the obturator coupler is coaxial with the cannula coupler.

8. The medical implantation device of claim 7, wherein when an obturator is coupled to the obturator coupler and a cannula is coupled to the cannula coupler, translation of the actuator towards the proximal end of the housing causes the obturator to be received within the cannula.

9. The medical implantation device of claim 1, wherein the obturator coupler is integrally formed within the housing.

10. The medical implantation device of claim 1, wherein the device is composed of a light-weight material.

11. The medical implantation device of claim 10, wherein the light-weight material comprises a plastic.

12. The medical implantation device of claim 1, wherein the device is sterilizable and reusable.

13. The medical implantation device of claim 1, wherein the cannula coupler defines a washer having a washer slot configured to receive the proximal end of the cannula.

14. A device for implanting a medical device into a patient, comprising:
a housing having a proximal end and a distal end;
an actuator slidably coupled to the housing and translatable along the housing parallel to an implantation axis;
a guide track disposed on the housing and configured to receive and retain the actuator, wherein the guide track is shaped to limit radial movement and rotation of the actuator relative to the implantation axis; and
a latch arm coupled to one of the housing or the actuator, the latch arm being movable between a closed position and an open position, wherein in the closed position, the latch arm engages with the other of the housing or the actuator to limit axial movement of the actuator relative to the implantation axis, and wherein in the open position, the latch arm allows the actuator to translate along the guide track.

15. The device of claim 14, wherein the guide track comprises a pair of rails extending parallel to the implantation axis along a track sidewall of the housing.

16. The device of claim 14, wherein the latch arm comprises arm teeth configured to engage with corresponding teeth on the other of the housing or the actuator.

17. The device of claim 14, wherein the latch arm is configured to limit distal movement of the actuator while permitting proximal movement of the actuator when in the closed position.

18. The device of claim 14, wherein the guide track comprises a cap opposite a track sidewall, the cap connecting a pair of rails and configured to mitigate radial translation of the actuator relative to the implantation axis.

19. The device of claim 14, wherein the latch arm is biased towards the closed position by a spring.

20. A device for implanting a medical device into a patient, comprising:
a housing having a proximal end and a distal end;
an obturator removably coupled to the distal end of the housing;
an actuator slidably coupled to the housing and translatable along the housing parallel to an implantation axis; and
a cannula removably coupled to a distal end of the actuator, wherein the cannula is configured to receive the obturator and the medical device, and wherein translation of the actuator causes the cannula to slide over the obturator to release the medical device through a distal opening of the cannula.
